SEQUENCE LISTING (31 sequences)

SEQ ID NO: 1 - DNA, 140 bp, Homo sapiens
agacctgccctgcagtgattgcctgtagctctccaggcatcaacggcttcccaggcaaagatgggcgtgatggcaccaagggagaaaagggggaaccaggtacgtgttgggctgttctgtctctgcaattctttaccttc

SEQ ID NO: 2 - DNA, 25 bp, Artificial Sequence
  MBP primer
tgattgcctgtagctctcaaggcat

SEQ ID NO: 3 - DNA, 28 bp, Artificial Sequence
  MBP primer
ggtaaagaattgcagagagacgaacagc

SEQ ID NO: 4 - DNA, 21 bp, Artificial Sequence
  MBP probe
caggcaaagatgggcgtgatg

SEQ ID NO: 5 - DNA, 21 bp, Artificial Sequence
  MBP probe
caggcaaagatgggtgtgatg

SEQ ID NO: 6 - DNA, 21 bp, Artificial Sequence
  MBP probe
caggcaaagatgggagtgatg

SEQ ID NO: 7 - DNA, 21 bp, Artificial Sequence
  MBP probe
caggcaaagatgggggtgatg

SEQ ID NO: 8 - DNA, 22 bp, Artificial Sequence
  MBP probe
tgatggcaccaagggagaaaag

SEQ ID NO: 9 - DNA, 22 bp, Artificial Sequence
  MBP probe
tgatgacaccaagggagaaaag

SEQ ID NO: 10 - DNA, 22 bp, Artificial Sequence
  MBP probe
tgatgtcaccaagggagaaaag

SEQ ID NO: 11 - DNA, 22 bp, Artificial Sequence
  MBP probe
tgatgccaccaagggagaaaag

SEQ ID NO: 12 - DNA, 22 bp, Artificial Sequence
  MBP probe
tgatggcaccaagggagaaaag

SEQ ID NO: 13 - DNA, 22 bp, Artificial Sequence
  MBP probe
tgatggcaccaaggaagaaaag

SEQ ID NO: 14 - DNA, 22 bp, Artificial Sequence
  MBP probe
tgatggcaccaaggtagaaaag

SEQ ID NO: 15 - DNA, 22 bp, Artificial Sequence
  MBP probe
tgatggcaccaaggcagaaaag

SEQ ID NO: 16 - DNA, 23 bp, Artificial Sequence
  IL-1 beta primer
aaattttgccacctcgcctcacg

SEQ ID NO: 17 - DNA, 23 bp, IL-1 beta primer
agtcccggagcgtgcagttcagt

SEQ ID NO: 18 - DNA, 24 bp, Artificial Sequence
  Il-1 beta probe
tcttcttcgacacatgggataacg

SEQ ID NO: 19 - DNA, 24 bp, Artificial Sequence
  IL-1 beta probe
tcttctttgacacatgggataacg

SEQ ID NO: 20 - DNA, 24 bp, Artificial Sequence
  IL-1 beta probe
tcttcttagacacatgggataacg

SEQ ID NO: 21 - DNA, 24 bp, Artificial Sequence
  IL-1 beta probe
tcttcttggacacatgggataacg

SEQ ID NO: 22 - DNA, 24 bp, Artificial Sequence
  Lymphotoxin primer
cttctctgtctctgactctccatc

SEQ ID NO: 23 - DNA, 20 bp, Artificial Sequence
  Lymphotoxin primer
caaggtgagcagagggagac

SEQ ID NO: 24 - DNA, 21 bp, Artificial Sequence
  Lymphotoxin probe
ttctgccatgattcctctctg

SEQ ID NO: 25 - DNA, 21 bp, Artificial Sequence
  Lymphotoxin probe
ttctgccatggttcctctctg

SEQ ID NO: 26 - DNA, 21 bp, Artificial Sequence
  Lymphotoxin probe
ttctgccatgtttcctctctg

SEQ ID NO: 27 - DNA, 21 bp, Artificial Sequence
  Lymphotoxin probe
ttctgccatgcttcctctctg

SEQ ID NO: 28 - DNA, 25 bp, Artificial Sequence
  TNF alpha primer
gttagaaggaaacagaccacagacc

SEQ ID NO: 29 - DNA, 19 bp, Artificial Sequence
  TNF alpha primer
tcctccctgctccgattcc

SEQ ID NO: 30 - DNA, 17 bp, Artificial Sequence
  TNF alpha probe
gcatggggacggggttc

SEQ ID NO: 31 - DNA, 17 bp, Artificial Sequence
  TNF alpha probe
gcatgaggacggggttc

(19) United States
(12) Patent Application Publication
(10) Pub. No.: US 2004/0058317 A1
(43) Pub. Date: Mar. 25, 2004

(54) Single nucleotide polymorphic discrimination by electronic dot blot assay on semiconductor microchips

(76) Inventors: Patrick N. Gilles, Carlsbad, CA (US); Patrick J. Dillon, Carlsbad, CA (US); David J. Wu, La Jolla, CA (US); Charles B. Foster, Silver Spring, MD (US); Stephen J. Chanock, Bethesda, MD (US)

Correspondence Address: O'MELVENY & MEYERS, 114 PACIFICA, SUITE 100, IRVINE, CA, 92618, US

(21) Appl. No.: 09/727,030

(22) Filed: Nov. 30, 2000

Related U.S. Application Data
(63) Continuation of application No. PCT/US00/08617, filed on Mar. 28, 2000.
(60) Provisional application No. 60/126,865, filed on Mar. 30, 1999.

(51) Int. Cl. C12Q 1/68

(52) U.S. Cl. 435/6

(57) ABSTRACT

A rapid assay for single nucleotide polymorphism (SNP) detection that utilizes electronic circuitry on silicon microchips is disclosed. The method provides accurate discrimination of amplified DNA samples following electronic assisted transport, concentration, and attachment of DNA to selected electrodes (test sites). The test sites make up organized arrays of samples that are distinguished by using internal controls of dual labeled reporters comprising wild-type and mismatched sequences to validate the SNP genotype. This method has been used to discriminate the complex quadra-allelic SNP of mannose binding protein.

### RELATED APPLICATION INFORMATION

This application is a continuation of International Application No. PCT/US00/08617, with an international filing date of Mar. 28, 2000, entitled “SINGLE NUCLEOTIDE POLYMORPHIC DISCRIMINATION BY ELECTRONIC DOT BLOT ASSAY ON SEMICONDUCTOR CHIPS,” which claims priority from U.S. Provisional Application No. 60/126,865, filed Mar. 30, 1999, also entitled “SINGLE NUCLEOTIDE POLYMORPHIC DISCRIMINATION BY ELECTRONIC DOT BLOT ASSAY ON SEMICONDUCTOR CHIPS.”

### FIELD OF THE INVENTION

This invention relates to the detection of single nucleotide polymorphisms (SNPs). More specifically, this invention relates to detecting SNPs using electronically addressable microchips.

### BACKGROUND

The following description provides a summary of information relevant to the present invention. It is not an admission that any of the information provided herein is prior art to the presently claimed invention, nor that any of the publications specifically or implicitly referenced are prior art to that invention.

Single nucleotide polymorphisms (SNPs) are point mutations that constitute the most common type of genetic variation and are found at a rate of 0.5-10 per every 1000 base pairs within the human genome. SNPs are stable mutations that can be contributory factors for human disease and can also serve as genetic markers. The complex interaction between multiple genes and the environment necessitates the tracking of SNPs in large populations in order to elucidate their contribution to disease development and progression. Current efforts are underway in identifying human SNPs through large-scale mapping projects with high density arrays, mass spectrometry, molecular beacons, peptide nucleic acids, and the 5′ nuclease assay. However, technologies such as these are not yet widely used in research and clinical settings.

Conventional methods for SNP genotyping have depended on either (1) gel-based sequencing of PCR amplified material such as that described by Kornman et al. (J. Clin. Peridontol. V.24, pp.72-77 (1997), (2) restriction fragment length polymorphisms (RFLP) such as that described by A. J. Jeffreys (Cell V.18, pp.1-10 (1979), (3) dot blot hybridization with allele-specific oligonucleotide probes (ASO) such as that described by Malmgren et al. (Clin. Genet. V.50, pp.202-205 (1996), or (4) single-strand conformational polymorphism (SSCP) as described by Schafer et al. (Nat. Biotechnol. V15, pp.33-39 (1995). Sequencing procedures, although effective, can be time consuming, and artifacts caused by secondary structures may be encountered. RFLPs generally encompass only a subset of SNP polymorphisms. ASO may require protracted heat denaturation steps, and SSCP is less amenable to automation. Passive hybridization to high density oligonucleotide arrays has accomplished large-scale genotyping of SNPs. However, the sites on conventional DNA arrays cannot be individually controlled and the same process steps must be performed over the entire array.

We present a method which advances the art of SNP detection by integrating microelectronics with the molecular biological arts wherein the manipulation of an electric field on a microchip permits the rapid concentration, hybridization, and detection of DNA molecules at designated test sites. This new method further uses polarity reversal at the test sites for denaturing single base pair mismatched oligonucleotides as well as a variety of test site configurations to take advantage of flexibility of design for rapid throughput screening of genes of interest.

### SUMMARY OF THE INVENTION

The current invention provides numerous aspects of which a preferred embodiment comprises the use of a bio-electronic microchip with nucleic acid hybridization in the detection of single nucleotide polymorphisms (SNPs) in single or multi-allelic gene complexes and other SNP containing nucleic acids including, but not limited to, the presence of multiple polymorphic sites within the same haplotype of a nucleic acid, the complex quadra-allelic SNP of mannose binding protein, the Fc-gamma receptors, the major histocompatibility complex, and the SNP-typing of Interleukin 1β.

In another embodiment, the invention comprises the multiplex assaying of SNPs from a multiplicity of patient samples containing nucleic acid sequences of interest, such as the ability to rapidly SNP genotype a large number of samples for a subset of genes in an on-demand basis.

In another embodiment, the invention comprises the ability to simultaneously screen multiple SNPs from different genes from a single patient sample.

In a further embodiment, the invention comprises the use of allele-specific probes wherein polymorphic discrimination is attained equally well regardless of whether the polymorphic nucleotide is located 5′, centrally, or 3′ within the allele-specific probe.

In another embodiment, the method uses labeling of one strand of an amplified target nucleic acid wherein the label (including, but not limited to, a label comprising biotin) is incorporated on the amplification primer used in the amplification reaction to amplify said target nucleic acid.

In yet another embodiment, the method includes the binding of a labeled target amplification product to a specified test site on an electronically addressable microchip.

In still another embodiment, the method includes the capacity to monitor various stages of electronic hybridization and electronic stringency in real time thereby providing validated control during assaying.

In another embodiment, the method uses a fluorescence hybridization pattern scoring method based on Mean Fluorescent Intensity per second (MFI/s) values comprising criteria that compares the magnitude of signal differences between positive and negative samples such that any signal scored as negative or positive is statistically clearly below or above, respectively, the intensity of the MFI observed for the positive or negative, respectively. Variable scoring criteria may also be carried out by comparison of the MFI/s obtained from signals resulting from binding of negative vs. positive reporter probes when applied to individual samples. In this case, the samples are tested using at least two different reporter probes that are designed to hybridize to specific targets in the sample. Results obtained from scoring also depend upon the nature of the permeation layer and the densities of functional components therein such as concentration of avidin, or other binding moieties, or composition of the permeation layer such as agarose, or hydro gel.

Additionally, the method of the invention further comprises advantages of addressable microchips over passive array technologies, including;

a. the capacity to individually control test sites thereby allowing custom configuration of test arrays immediately prior to testing;

b. the capacity to electronically address (i.e., transport), concentrate, and hybridize nucleic acid molecules in a time period of seconds;

c. the capacity to control electronic stringency at specified test sites thereby permitting the simultaneous use of unrelated molecules on the same open array microchip;

d. the capacity of the disclosed methodology to be applied toward use in automated loaders for arrays comprising hundreds of test sites; and

e. the capacity of the disclosed technology to be used in analyzing RNA expression in a limited number of cells.

### BRIEF DESCRIPTION OF THE DRAWINGS

FIG. 1 is a photomicrograph of one microelectronic array format design.

FIG. 2 is a schematic of an electronic dot blot assay.

FIG. 3 represents a DNA sequence (SEQ ID No: 1) of the sense strand of wild-type human mannose binding protein (MBP) from nucleotides 1001 to 1158 denoted allele A. The SNP bases and positions for allele B, C, and D are noted. The figure further shows positions of forward (MBL F) and reverse (R-1120) amplification primers (arrows). Each reporter probe is synthesized with either a cy3 or cy5 label.

FIGS. 4A, 4B, 4C, and 4D are photomicrographs of SNP detection results on a microchip wherein each row of four capture sites were loaded with one of four blinded amplified MBP samples (NC47, NC48, NC49, or NC50). A central fifth row was loaded with a non-specific (NS) thiopurine s-methyltransferase (TPMT) amplicon. The figures show hybridization of allele-specific reporter probes labeled with either cy3 or cy5 before (FIGS. 4A and 4B) and after (FIGS. 4C and 4D) electronic stringency. FIGS. 4A and 4C detect cy3 fluorescence while FIGS. 4B and 4D detect cy5 fluorescence.

FIGS. 5A and 5B are charts showing quantification of the emitted fluorescence from sample NC49 for the cy3 image (FIG. 5A) and cy5 image (FIG. 5B) of FIG. 4. Sample NC49 is identified as an A/A homozygote.

FIGS. 6A and 6B are charts showing quantification of the emitted fluorescence from sample NC50 for the cy3 image (FIG. 6A) and cy5 image (FIG. 6B) of FIG. 4. Sample NC50 is identified as an A/B heterozygote.

FIGS. 7A and 7B are charts showing quantification of the emitted fluorescence from sample NC47 for the cy3 image (FIG. 7A) and cy5 image (FIG. 7B) of FIG. 4. Sample NC47 is identified as an B/B homozygote.

FIGS. 8A and 8B are photomicrographs showing cy5 detection results on a microchip for samples containing A and D MBP alleles. Sample LM18 is a D/D homozygote. Sample LM27 is an A/D heterozygote. The remaining samples shown are A/A homozygotes.

FIG. 9A is a graph showing quantification by detection of cy3 label of the IL-1β T/T amplicon hybridized with allele C, allele T, and mismatched reporter groups versus increasing amperage of electronic stringency.

FIG. 9B is a photomicrograph showing detection results for the final stringency shown in FIG. 9A.

FIG. 10A is a graph showing quantification by detection of cy5 label of the IL-1β T/T amplicon hybridized with allele C and allele T groups versus increasing amperage of electronic stringency.

FIG. 10B is a photomicrograph showing detection results for the final stringency shown in FIG. 10A.

FIGS. 11A and B are bar graphs showing the positive and negative signals of probe with A/A and G/G homozygotes of the lymphotoxin gene respectively.

FIGS. 12A and B are bar graphs showing the positive, negative and control signals of probe with targets in the Tumor Necrosis Factor a gene.

FIGS. 13A to L show a series of photographs of microarrays wherein the invention with respect to multiplex analysis is demonstrated.

### DETAILED DESCRIPTION OF THE INVENTION

Definitions

As used herein, “mean fluorescent intensity” (MFI) is the average of the pixel count values over a region of interest upon normalizing the camera integration time to one second. The value ranges will vary according to the makeup of the permeation layer.

As used herein, “fluorescence hybridization scoring” refers to the designation of hybridization patterns post stringency wherein scoring is based on criteria that may vary according to the nature of the permeation layer and its functional components. In one embodiment of the invention, the criteria specify that (1) the mismatched probes be less than 10 MFI/second; (2) any allele-specific reporter less than 25 MFI/s be scored as a negative; and (3) any reporter greater than 30 MFI/s be scored as positive. This scoring criteria represents an example of how signal parameters would be used to identify single nucleotide polymorphisms using the electronic dot blot assay and are not intended to limit the invention in any way. The magnitude of the MFI/s in designating a positive or negative score could be set at an appropriate level suitable to a different level of detection depending upon the variable criteria.

Additionally, the used of multiple reporter hybridizations (or internal redundancy) per sample was designed for genotypic determination independent of comparison with a replicate or control sample.

Detailed Embodiments

The current invention, among other aspects, provides a method of carrying out a high throughput assay for detecting single nucleotide polymorphisms (SNPs) in multi-allelic gene complexes and other SNP containing nucleic acids including, but not limited to the presence of multiple polymorphic sites within the same haplotype of a nucleic acid, the complex quadra-allelic SNP of mannose binding protein, the Fc-gamma receptors, the major histocompatibility complex, and the SNP-typing of Interleukin 1β, using an electronically addressable bio-electronic microchip. As explained herein, the integration of microelectronics with nucleic acid hybridization can rapidly and accurately discriminate between SNP sequences. In fact, the dual fluorescent electronic dot blot assay of the invention has proven 100% accuracy with respect to the detection of SNPs in 22 blinded MBP quadra-allelic samples and 13 blinded samples that were enhanced for the D allele. This accuracy is derived by the novel use of mismatched oligonucleotides to validate the electronic microenvironment resulting in accurate discrimination of heterozygous sequences, and the internal redundancy imparted by cy3 and cy5 labeled reporters that confirmed the SNP sequence in individual samples. This ability to rapidly SNP genotype a large number of samples for a subset of genes in an on-demand basis is a significant advantage over existing methodologies.

Additionally, the use of semiconductor microelectronics for the transport and concentration of nucleic acids imparts several advantages over passive array technologies, including, but not limited to, (1) flexibility, wherein the open architecture and capacity to individually control test sites allows custom configuration of each array immediately prior to testing; (2) speed, wherein electronic addressing and electronic hybridization enable the transport, concentration, and hybridization of DNA molecules in seconds rather than hours; (3) multiplexing, wherein the ability to control electronic stringency at individual test sites permits the simultaneous use of unrelated molecules on the same microchip; (4) efficiency, wherein the ability to monitor various stages of electronic hybridization and electronic stringency in real time provides a more validated control during the assay; (5) laboratory-on-a-chip technology, wherein the application of electronics provides a means for automation and elimination of time-consuming, up-front processes by employing dielectrophoresis as a means of cell concentration, disruption, and nucleic acid concentration/amplification directly on the microchip; (6) automation, wherein automated loaders for 100-pad and 400-pad semiconductor chips may be used to achieve high throughput parameters; and (7) gene expression, wherein the technology described here may be applied to analysis of RNA expression from a small number of cells.

### EXAMPLE 1

### MBP Allele Detection

The human mannose binding protein (MBP or MBL2) is an important component of the innate immune system and is capable of opsonizing pathogenic microorganisms. MBP is particularly important in children who have not yet developed immunity to many pathogens. Inheritance of any of several common variant forms of MBP gives rise to a subtle immunologic defect that can be enhanced during a period of immunosuppression. Four distinct alleles of the MBP gene have been identified. The potential clinical relevance of MBP and its genetic complexity made this sequence a relevant target for analysis by the method of the invention.

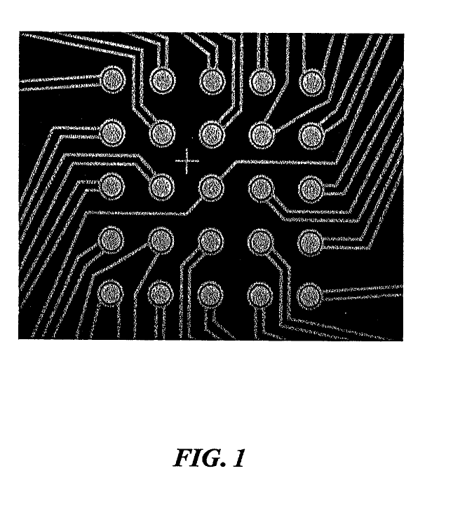

The method of the invention uses an electronically addressable microchip manufactured by Nanogen, Inc., (San Diego, Calif.). The microchips were fabricated on oxidized silicon wafers using standard process technology. The wafer was coated with 20 nm of titanium and 100 nm of platinum using a radio frequency sputter process. Using standard photolithographic techniques, the metal layer was patterned and etched to form the electrode array. A silicon oxide insulating layer was then deposited by plasma-enhanced chemical vapor deposition over the entire wafer. The exposed electrode diameter is 80 μm and the center-to-center spacing is 200 μm. The wafers were coated with photoresist and diced into 1 cm square chips. In the current example, the 1 cm square chips comprised 25 microelectrodes arranged in a five-by-five array (FIG. 1). Each electrode or test site may be individually charged positive, negative, or neutral for the movement and concentration of molecules to and from the test site.

An agarose permeation layer containing streptavidin is used to coat the electrode containing chip surface thereby separating the biological material of samples from the harsh electrochemical environment near the electrode and further allowing binding of biotinylated nucleic acid of the sample to the chip surface above the electrode. The permeation layer coating is applied by employing glyoxal agarose (FMC Bioproducts, Rockland Me.) combined with streptavidin (5 mg/ml, Boehringer Mannheim, Indianapolis, Ind.) to yield a 2% agarose and 1 mg/ml streptavidin mixture. The chips are spin coated with the streptavidin-agarose solution and schiff base linkages are reduced with 0.2 M sodium cyanoborohydride/0.3 M sodium borate, pH 9.0 for 60 min.

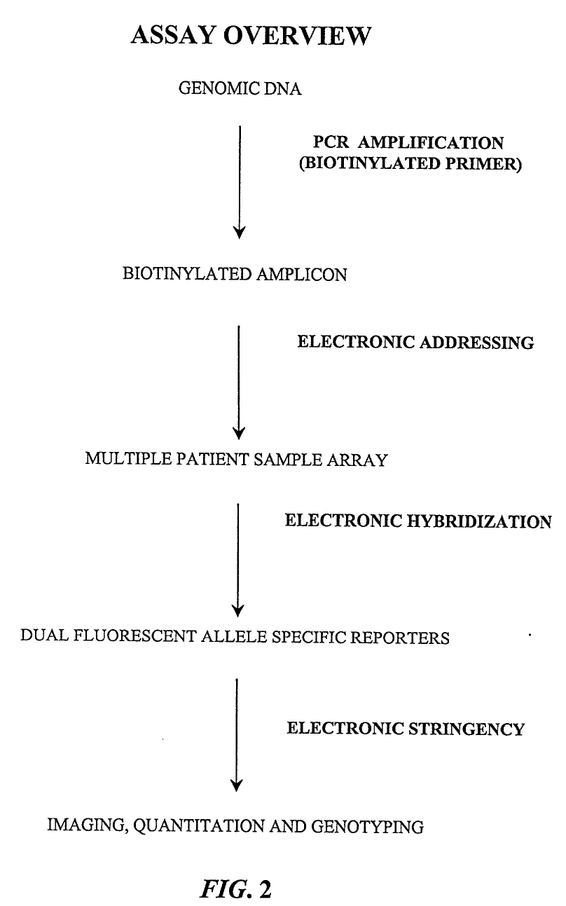

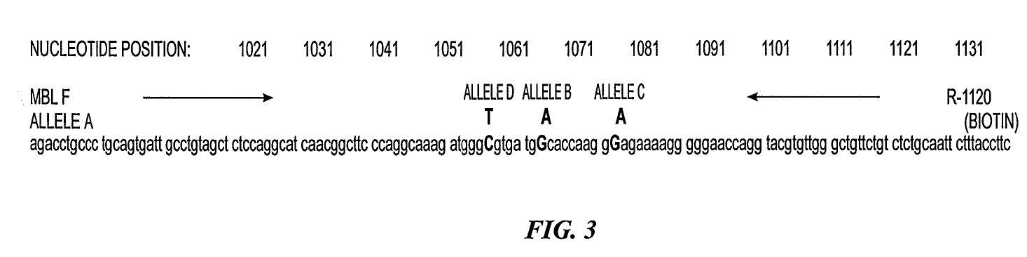

The multiple SNPs of the MBP gene in question occur within a 17 base pair region of the gene. The wild-type sequence of the MBP gene is defined as allele A while SNP allele types are designated as alleles B, C, and D. As shown in FIG. 3, a 123 base fragment encompassing this polymorphic region may be amplified from patient genomic nucleic acid samples and used in the method of the invention. FIG. 2 shows one embodiment of the method assay steps.

Primers for amplifying the MBP gene (Genbank HSMBP1A-X15954) were designed such that the sense strand primer comprised the nucleotide sequence 5′-TGATTGCCTGTAGCTCTCCAGGCAT-3′ (SEQ ID No: 2) while the reverse primer comprised the nucleotide sequence, biotin-5′-GGTAAAGAATTGCAGAGAGACGAACAGC-3′ (SEQ ID No: 3) (i.e., the 5′ end of the reverse primer is biotinylated).

The 123 base fragment in the method of this example was amplified from patient genomic DNA samples by PCR wherein the reaction mixture comprised 2-4 μl of DNA, 1×PCR buffer II (Perkin-Elmer, Branchburg, N.J.), 1.5 mM MgCl₂, 200 μM dNTPs, 200 μM of each primer, 2.0 U of AmpliTaq Gold (Perkin-Elmer), and 280 nM of Taq Start Antibody (Clontech, Palo Alto, Calif.), in a 100 μl reaction. The reaction mixes were cycled at 95° C. for 10 min. (one cycle), 35 cycles at 95° C. for 30 seconds, 58° C. for 60 seconds, and 72° C. for 120 seconds, followed by incubation at 72° C. for 12 minutes in a 9700 Thermocycler (Perkin-Elmer). Each DNA sample was purified by Qiagen column (Valencia, Calif.), resuspended in water and quantified by gel electrophoresis using a DNA mass ladder control (Gibco BRL, Gathersburg, Md.). The DNA samples were resuspended at a concentration of 2.5-10 nM in 100 mM histidine (Sigma, St. Louis, Mo.).

The samples (approximately 40 to 400 μl in volume) were heat denatured for 2-10 minutes at 95° C. and quick cooled on ice. 35 μl of sample were applied to the microchip and electronically transported (addressed) using positive bias direct current to a column of four positively charged test sites at 400 nA/test site for 120 seconds. The unattached DNA was removed by washing with histidine buffer. This step was repeated for each sample. An optional procedure of treating the completely addressed array for 5 minutes with 0.5×SSC, pH 11.5 followed by extensive washing with water and histidine yielded greater hybridization signals. The addressed amplicons remained attached to their respective test sites through interaction with the streptavidin previously embedded in the permeation layer.

The nucleic acid at each test site was then hybridized to mixtures of fluorescently labeled allele-specific reporter oligonucleotide probes by electronic hybridization. The reporter probes (see Table 1) were synthesized with either a cy3 or cy5 fluorophore linked at the 5′ end (BioServe Biotechnologies, Laurel, Md.). Electronic hybridization was carried out such that wild-type and SNP cy3 and cy5 reporter groups were resuspended in a range of between 75 to 125 nM each in 100 mM histidine buffer. The two cy3 labeled mismatched reporters of each allele group were mixed equally at 37.5 nM to yield a combined 75 nM in buffer. Each reporter group (20-35 μl) was applied to the microchip and hybridized to a row of captured amplicons at 475 nA/test site for 15 seconds. Excess reporter was removed by washing with 100 mM histidine. The second, third, and fourth reporter groups were applied in the same row-wise manner. After hybridization, the chip was washed in 20 mM diabasic NaH₂PO₄, 20 mM Trisbase, pH 9.5, (20/20 buffer) for electronic stringency.

Single base pair mismatched reporter probes were then preferentially denatured by reversing the charge polarity at individual test sites with increasing amperage (electronic stringency). A pulsed current of 0.5 μA/test site 0.1 second on, 0.2 seconds off, for 150 cycles (or 0.1 second on, 0.1 second off, 50 cycles), was applied to each test site. The chip was washed in 20/20 buffer to remove the denatured reporter and imaged with IPLab software. Imaging and fluorescence were quantified as the amperage was ramped to completion. Specifically, currents of 0.6 μA/test site, 0.7 μA/test site, 0.8 μA/test site, 0.9 μA/test site, and 1.0 μA/test site were used. A biotinylated amplicon that spans the TPMT codon 460 polymorphism was used as a nonspecific background control. Each image was normalized to MFI/s and the nonspecific counts were subtracted from each MBP test site for final quantification. Positive test sites retained significant signal above the mismatched controls.

The invention method of this example used instrumentation wherein electronic connections to the microchip were made by an epoxy ring probe card (Cerprobe, Phoenix, Ariz.) mounted on a micromanipulator 6000 (Micromanipulator Company, Carson City, NV). The power supply (Keithley 236; Keithley Instruments, Cleveland, Ohio) sourced either fixed potential difference or a fixed current between its terminals through an array of relays (National Instruments, Austin, Tex.). Computer hardware (Macintosh Power PC, Apple Computer, Cupertino, Calif.) and IPLab Spectrum version 3.1.1 software (Signal Analytics Corporation, Vienna, Va.) allowed graphical user menu control of the individual array locations. Laser excitation was by a HeNe 633 nm laser (8 mW output; Research Electro-Optics, Boulder, Colo.) and a frequency-doubled diode pumped solid state laser (5 mW output; Laser Compact, Moscow) 532 nm. Fluorescence was observed through a 8× objective (numerical aperture 0.15) with the banded filters at 575 nm (for cy3) or 670 nm (for cy5) (Chroma Tech, Brattleboro, Vt.). The fluorescent signal was scanned and collected by a charged couple device camera (Princeton Instruments, Trenton, N.J.). The scanned image was quantified by IPLab Spectrum software.

As shown in Table 1, reporter probes specific for wild-type, a particular SNP allele sequence or a mismatch of wild-type and SNP were synthesized. The wild-type and SNP reporters were labeled with both cy3 (1,1′-bis(ε-carboxypentyl)-3,3,3′,3′-tetramethylindocarbocyanine-5,5′-disulfonate potassium salt di-N-hydroxysuccinimde ester) and cy5 (1,1′-bis(ε-carboxypentyl)-3,3,3′,3′-tetramethylindodicarbocyanine-5,5′-disulfonate potassium salt di-N-hydroxysuccinimde ester). The wild-type probes labeled with cy3 were kept separate from wild-types probes labeled with cy5. The SNP probes were treated the same. However, the wild-type and SNP labeled reporters were combined into two types of probe mixtures. Specifically, these mixtures or groups were (1) wild-type-cy3/SNP-cy5, and (2) wild-type-cy5/SNP-cy3. Mixing of the differentially labeled probes allowed their simultaneous hybridization to the amplified patient DNA samples at each test site with each hybridization event recorded in duplicate. For each SNP allele, two additional reporter oligonucleotide probes were created that incorporated a distinct mismatch of both wild-type and SNP. These were labeled only with cy3. Cy3 in this experiment was chosen arbitrarily for experimental convenience. However, any label could be attached for the purpose of distinguishing the alleles. These mismatched probes were combined also into two groups at equivalent molar concentration with either the bona fide wild-type or SNP probes labeled with cy5 ((1) wild-type-cy5/mismatch-cy3 and (2) SNP-cy5/mismatch-cy3). These additional reporter pairs permitted comparison of the interaction with matched and mismatched probe sequences even if both the wild-type and SNP sequences were present in the same sample (heterozygote).

TABLE 1

| | | |
|---|---|---|
| Type reporter | specificity of | |
| probe | reporter | reporter sequence |

| | | | |
|---|---|---|---|
| allele A or D | allele A, cy3 or cy5 | 5′caggcaaagatgggCgtgatg3′ | (SEQ ID No: 4) |
| discrimination | | | |
| | allele D, cy3 or cy5 | 5′caggcaaagatgggTgtgatg3′ | (SEQ ID No: 5) |
| | mismatch A, cy3 | 5′caggcaaagatgggAgtgatg3′ | (SEQ ID No: 6) |
| | mismatch D, cy3 | 5′caggcaaagatgggGgtgatg3′ | (SEQ ID No: 7) |
| allele A or B | allele A, cy3 or cy5 | 5′tgatgGcaccaaggGagaaaag3′ | (SEQ ID No: 8) |
| discrimination | | | |
| | allele B, cy3 or cy5 | 5′tgatgAcaccaaggGagaaaag3′ | (SEQ ID No: 9) |
| | mismatch A and B at | 5′tgatgTcaccaaggGagaaaag3′ | (SEQ ID No: 10) |
| | position 1073, cy3 | | |
| | mismatch A and B at | 5′tgatgCcaccaaggGagaaaag3′ | (SEQ ID No: 11) |
| | position 1073, cy3 | | |
| allele A or C | allele A, cy3 or cy5 | 5′tgatgGcaccaaggGagaaaag3′ | (SEQ ID No: 12) |
| discrimination | | | |
| | allele C, cy3 or cy5 | 5′tgatgGcaccaaggAagaaaag3′ | (SEQ ID No: 13) |
| | mismatch A and C at | 5′tgatgGcaccaaggTagaaaag3′ | (SEQ ID No: 14) |
| | position 1082, cy3 | | |
| | mismatch A and C at | 5′tgatgGcaccaaggCagaaaag3′ | (SEQ ID No: 15) |
| | position 1082, cy3 | | |

Using these assay conditions, a blinded study was conducted on genomic DNA samples of known MBP sequence previously determined by standard sequencing techniques. Of the samples tested, one set had been identified as either homozygous or heterozygous for the four separate alleles and served as controls. The genotypes of the remaining samples (22 samples) were unknown. These 22 samples were tested blind in the method of the current invention. Additionally, a second set of 13 blinded samples were tested for the presence of the D allele site.

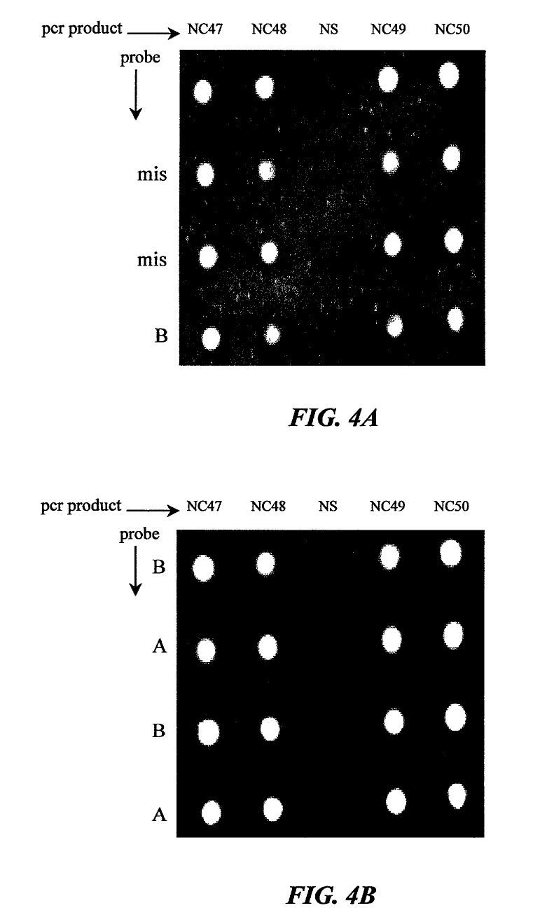

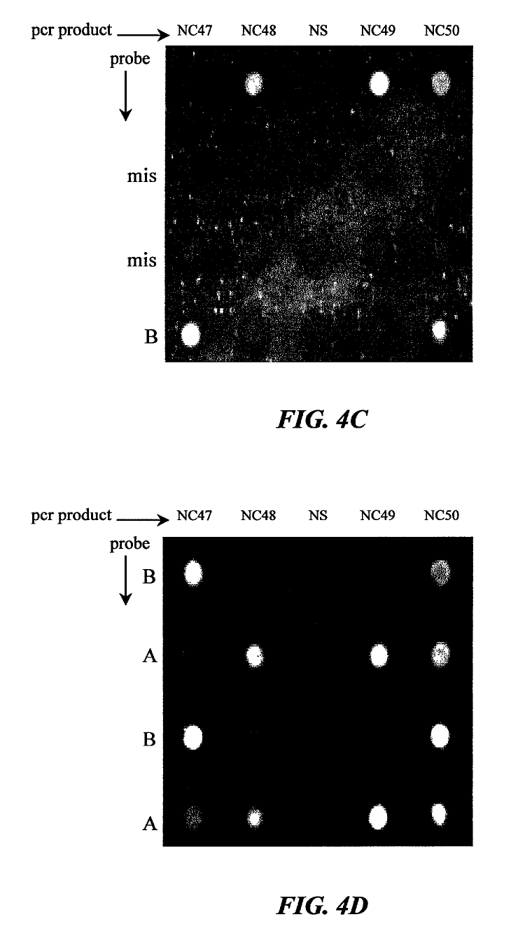

Each blinded amplicon was electronically addressed to a column of four test sites. Thus, a 25-site microchip array can accommodate four different patient samples and a nonspecific control amplicon. Each row of samples was hybridized with one of the four probe mixes (i.e. reporter groups (1) Acy3/Bcy5, (2) mismatch-cy3/Acy5, (3) mismatch-cy3/Bcy5, and (4) Bcy3/Acy5)). Each reporter group consisted of a cy3-labeled and a cy5-labeled SNP allele-specific oligonucleotide pair that differed by a single nucleotide mismatch. Images of a representative microchip containing four blinded samples (NC47, NC48, NC49, and NC50) after hybridization with the four cy3/cy5-labeled reporter groups specific for the A or B allele of MBP mentioned above are shown in FIGS. 4A-D. Electronic stringency was applied until the mismatched controls (mismatch-cy3) had been removed to background levels (FIG. 4C).

The distinct fluorescent patterns representative of the three possible genotypes (A/A, A/B, and B/B) are illustrated in FIGS. 4A-D. Analysis of sample NC47 (column 1) showed a discrete signal with the fourth reporter group (row 4) in the cy3 image (FIG. 4C) and signals with the first (row 1) and third (row 3) reporter groups in the cy5 image (FIG. 4D). Collectively, the signals were consistent with hybridization of reporter groups containing B allele-specific probes (representing a true match). Electronic stringency had reduced the signal from A allele-specific probes to background.

Blinded sample NC48 (FIG. 4C, column 2) displayed a cy3 signal with the first reporter group (row 1) and cy5 signal (FIG. 4D) with the second and fourth reporter groups (rows 2 and 4). Each signal correlated with hybridization of the A allele-specific reporters. Similarly, the column containing sample NC49 (column 4) showed a signal with the A allele-specific reporter in the cy3 image (FIG. 4C, row 1) and in the cy5 image (FIG. 4D, rows 2 and 4). Note that the fluorescence emitted from each test site in rows 2 and 3 of FIG. 4C was reduced to background intensity. These test sites had been hybridized with the mismatch-cy3 labeled reporters, known to be mismatched with respect to both the A and B allele sequences. The elimination of the mismatched signal confirmed that the appropriate level of electronic stringency had been attained for SNP discrimination and that any remaining reporter signal constituted a perfect match.

Evaluation of blinded sample NC50 (column5) in each panel (FIG. 4) revealed both A and B allele-specific binding in the cy3 image (FIG. 4C, rows 1 and 4) and the cy5 image (FIG. 4D, rows 1-4). The absence of signal in test sites hybridized with the mismatched cy3 reporters (FIG. 4C, rows 2-3) confirmed the completion of electronic stringency and identified the presence of both the A and B allele sequences within the NC50 amplicon.

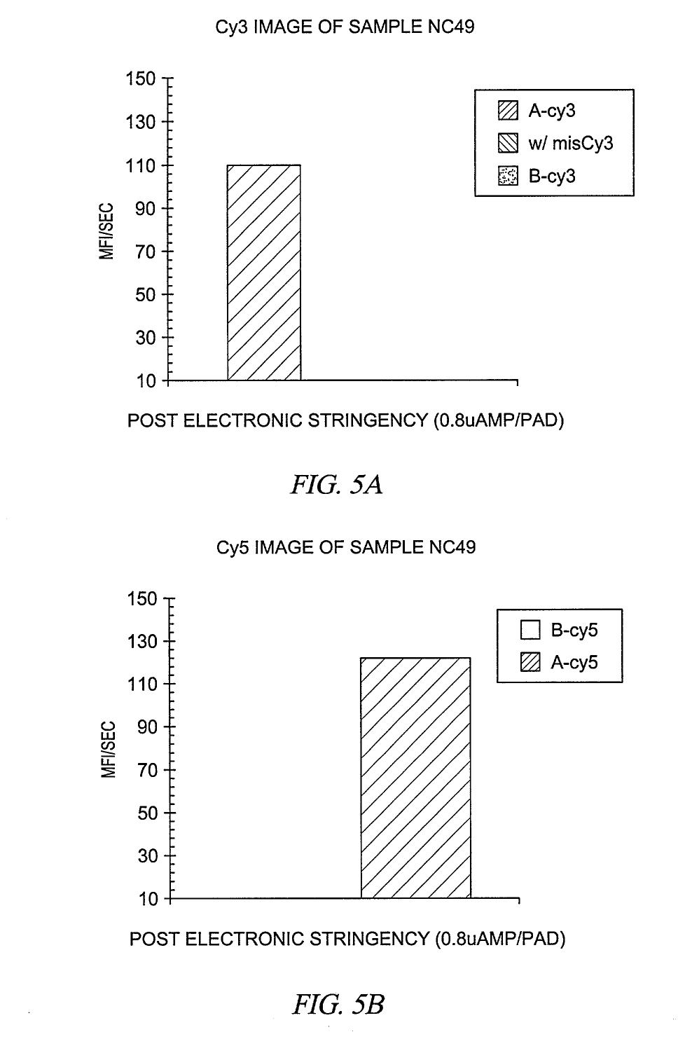

The above images were further discriminated for quantification of the emitted fluorescence from each sample. The binding of the MBP reporters to the nonspecific amplicon (NS) was approximately 4% of the total cy5 fluorescent intensity post-electronic hybridization and was twofold to fourfold higher with the cy3-labeled reporters prior to stringency. NC49 showed a greater than 10-fold discrimination in mean intensity between the A allele reporter with that of the B allele and mismatched reporters (FIGS. 5A and 5B). Thus, NC49 was scored as an A/A homozygote with respect to the B site. Similarly, sample NC48 was identified as an A/A homozygote with respect to the B site (data not shown).

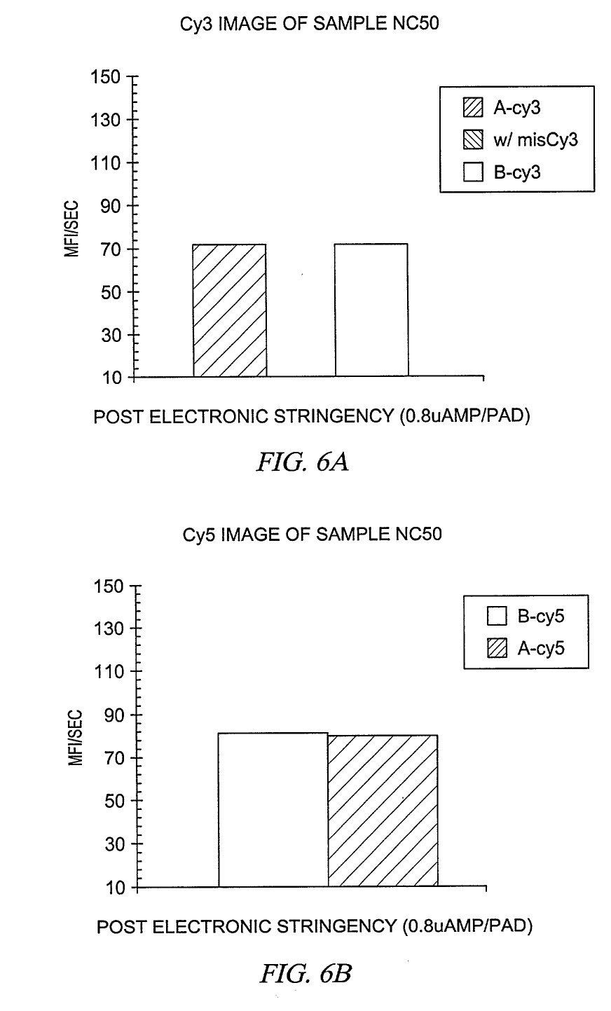

Quantification of blinded sample NC50, scored as A/B (FIGS. 6A and 6B) demonstrated that significant signal remained bound to the captured amplicon (approximately 70 MFI/s) with both A and B allele-specific cy3 reporters and cy5-labeled reporters. Thus, the NC50 amplicon was scored as an A/B heterozygote with respect to the B site.

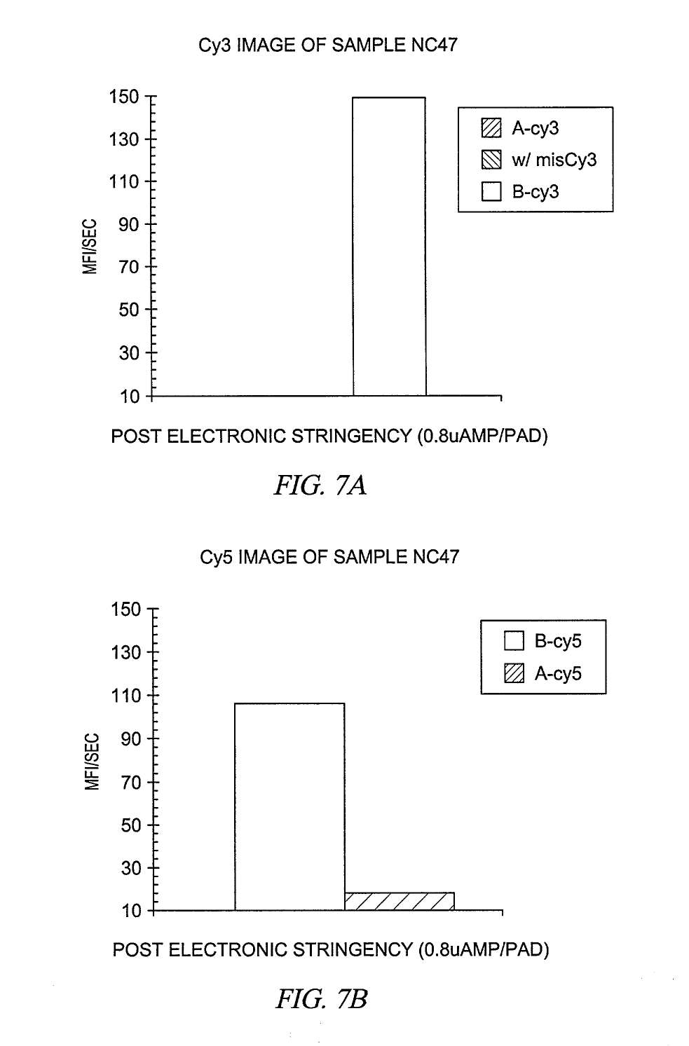

The NC47 sample fluorescence was quantified (FIGS. 7A and 7B) showing that the mismatched and A allele reporters are reduced to background intensity. Nevertheless, a signal of greater than 100 MFI/s was retained with the B allele reporter. Similarly, analysis of the cy5 image (FIG. 7B) revealed a reduction of the A allele signal (i.e., less than 25 MFI/s) with robust retention of B allele signal (i.e. greater than 100 MFI/s). Thus, NC47 was scored as a B/B homozygote.

Analysis of different blinded samples from patients scored as A/A with respect to the SNP alleles (sample population=18) demonstrated 100-fold higher binding of the A-cy3 reporter to the A/A samples compared with that of the mismatched cy3 reporters, and 26-fold greater binding than that of the B or C allele cy3-labeled SNP reporters (probe: mean±standard error of the mean (SEM); A-cy3: 200±36.2 MFI/s, SNP-cy3: 7.6±1.8 MFI/s, mismatch-cy3: 1.9±0.3 MFI/s). Conversely, analysis of seven different blinded samples scored as heterozygotes demonstrated that both the cy3-labeled A allele and SNP probes bound with near equal intensity to the samples as compared with the mismatched probes, which bound with 20-fold less intensity (probe: mean±SEM; A-cy3: 188.3±44.4 MFI/s, SNP-cy3: 150.1±42.3 MFI/s, mismatched-cy3: 7±2.2 MFI/s). equivalent ratios of matched to mismatched pairs were recorded with the cy5 labeled probes (data not shown).

Each of the 22 blinded samples was scored at the B and C allele positions. The nucleotide sequence at each location and A, B, and C genotypes were compared with the standard sequencing results (see Table 2). As shown, the microchip assay was in agreement with sequencing results on 44 of 44 SNP scores (22A/B calls and 22A/C calls) at the B and C sites. Thus, far, occurrence of the B and C allele sequences within the same haplotype has not been described. For the B/B genotype, only the perfectly matched B and C allele probes remained bound after stringency. Consequently, they had to be scored against both the B and C reporters before the compound heterozygote determination could be made. Although probes that span multiple variant sites may accurately discriminate between genotypes, the presence of multiple polymorphic sites within the same haplotype may confound discrimination performed in this way. In such cases, separate probe (10-24 nucleotide) sets for each polymorphic site would be advantageous. As demonstrated herein, polymorphic discrimination was attained equally well, regardless of whether the polymorphic nucleotide was located 5′, centrally, or 3′ within the allele-specific probe.

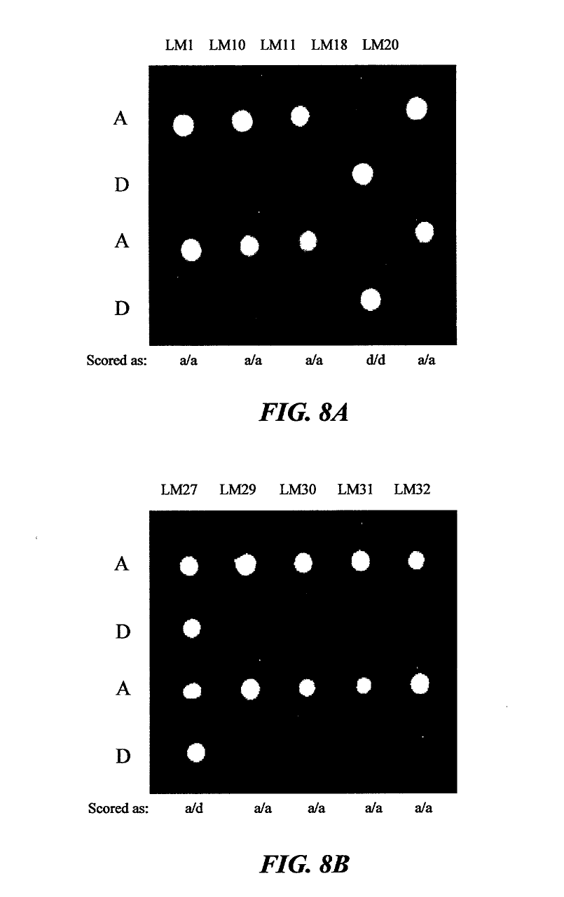

The 22 samples were subsequently reblinded and scored for the presence of the D allele (Table 2). The electronic assay of the invention correctly identified the only D allele sample (NC52-A/D) present among the 22 samples. As the frequency of occurrence of the D allele is low and the effectiveness of any SNP discrimination assay is its capacity to identify alleles of low frequency, a second set of 13 blinded samples (weighted toward validation of the D allele system, samples LM1, LM10, LM11, LM18, LM20, LM27, LM29, LM30, LM31, and LM32) was tested by the electronic assay with the D allele reporter probes. The scoring of both groups of samples for the D allele (sample population=35) with the electronic assay was in 1100% agreement with the sequencing results. Microchip images from 10 of the 13 blinded samples are shown in FIGS. 8A and 8B.

TABLE 2
Validation of SNP-typing of blinded samples by electronic dot blot
(NGEN) with standard DNA sequencing (NIH).

| | | | | Genotype | | | |
|---|---|---|---|---|---|---|---|
| | NGEN | | | A, B, C, D | | | |
| Samples | A/B | A/C | A/D | NGEN | NIH | Agree | Disagree |
| 1. NC7 | a/a | a/a | a/a | a/a | A/A | + | |
| 2. NC11 | a/b | a/a | a/a | a/b | A/B | + | |
| 3. NC20 | a/a | a/c | a/a | a/c | A/C | + | |
| 4. NC44 | a/a | a/a | a/a | a/a | A/A | + | |
| 5. NC45 | a/a | a/a | a/a | a/a | A/A | + | |
| 6. NC36 | a/a | a/c | a/a | a/c | A/C | + | |
| 7. NC37 | a/a | a/a | a/a | a/a | A/A | + | |
| 8. NC38 | a/a | a/a | a/a | a/a | A/A | + | |
| 9. NC40 | a/b | a/a | a/a | a/b | A/B | + | |
| 10. NC42 | a/a | a/a | a/a | a/a | A/A | + | |
| 11. NC43 | a/a | a/a | a/a | a/a | A/A | + | |
| 12. NC46 | a/a | a/a | a/a | a/a | A/A | + | |
| 13. NC47 | b/b | a/a | a/a | b/b | B/B | + | |
| 14. NC48 | a/a | a/a | a/a | a/a | A/A | + | |
| 15. NC49 | a/a | a/a | a/a | a/a | A/A | + | |
| 16. NC50 | a/b | a/a | a/a | a/b | A/B | + | |
| 17. NC51 | b/b | c/c | a/a | b/c | B/C | + | |
| 18. NC52 | a/a | a/a | a/d | a/d | A/D | + | |
| 19. NC53 | a/a | a/a | a/a | a/a | A/A | + | |
| 20. NC54 | a/b | a/a | a/a | a/b | A/B | + | |
| 21. NC55 | a/b | a/a | a/a | a/b | A/B | + | |
| 22. NC56 | a/b | a/a | a/a | a/b | A/B | + | |
| | | | | | | 100% | 0% |

### EXAMPLE 2

### Interleukin 1β Detection

Applicability of the current invention to detect SNPs was further demonstrated for the bi-allelic Interleukin 1β (IL-1β) polymorphism. The IL-1β sequence was retrieved from Genbank (XO4500) and contained a C to T SNP transition at position 5888. Primer sequences for use in PCR amplification comprised the sense strands nucleotide sequence, 5′-AAATTTTGCCACCTCGCCTCACG-3′ (SEQ ID No: 16) and the reverse strand nucleotide sequence, biotin-5′-AGTCCCGGAGCGTGCAGTTCAGT-3′ (SEQ ID No: 17) (i.e., the reverse strand was biotinylated).

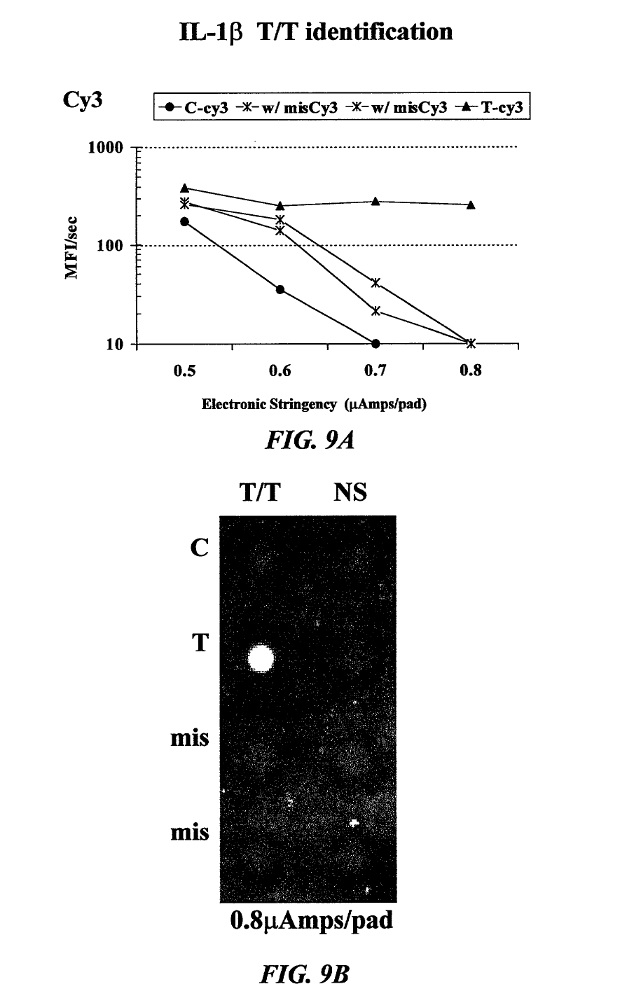

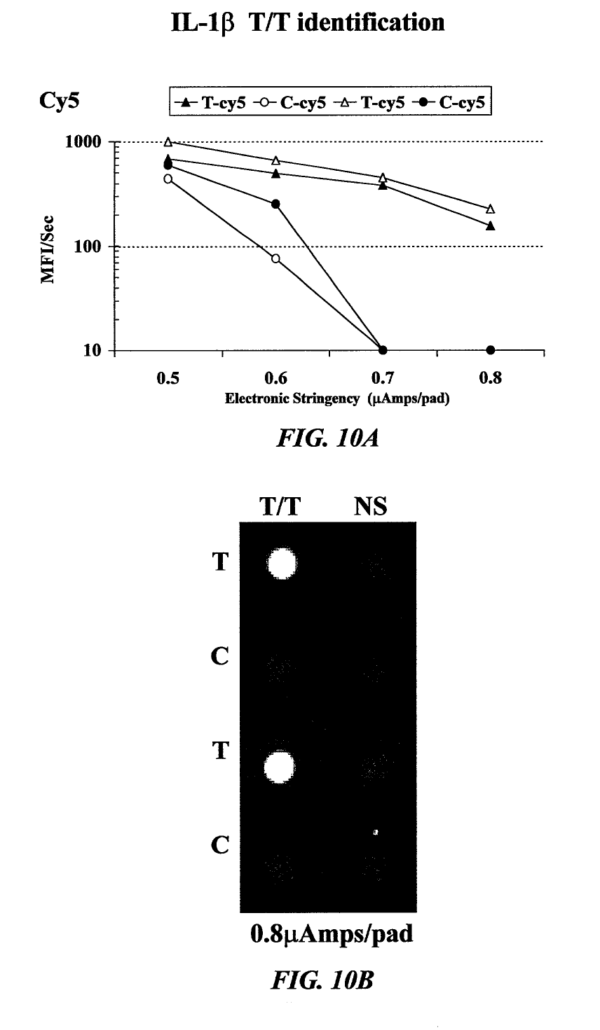

In this example, as described above, cy3 and cy5 labeled reporter groups were designed to identify the wild-type (C) and SNP variant (T) alleles (synthesis by Bioserve, Laurel, Md.). (see Table 3) Loss of fluorescence following increased electronic stringency illustrated the denaturation of the non-C/non-T mismatched (mis-cy3) reporters from test sites containing the known homozygous T/T biotinylated amplicon (FIGS. 9-10). Similarly, the cy3 and cy5 C allele reporters showed no evidence of stringent hybridization to the T/T amplicon. In contrast, a distinct signal of greater than 100 MFI/s was emitted from hybridization with the cy3 labeled T allele reporter (FIG. 9A) and cy5 labeled reporters (FIG. 10A). Thus, the amplicon known to be a T/T homozygote by sequencing technology was similarly scored as a T/T homozygote by the electronic format. In general, gradual decreases in overall signal intensity during electronic stringency was observed for both cy3 and cy5 reporters (data not shown).

Experiments, as shown in Example 5, demonstrate that SNP discrimination was obtained from an MBP, IL-1β, and TNF α multiplexed sample, in which the microchip was electronically stripped and reprobed for the genotyping of the second gene in the duplex.

TABLE 3

| | |
|---|---|
| reporter specificity | reporter sequence |

| | | |
|---|---|---|
| allele C | 5′tcttcttCgacacatgggataacg3′ | (SEQ ID No: 18) |
| allele T | 5′tcttcttTgacacatgggataacg3′ | (SEQ ID No: 19) |
| mismatch - A | 5′tcttcttAgacacatgggataacg3′ | (SEQ ID No: 20) |
| mismatch - G | 5′tcttcttGacacatgggataac 3′ | (SEQ ID No: 21) |

### EXAMPLE 3

### Lymphotoxin Gene Detection

Another demonstration of the current invention to detect SNPs was observed using the Lymphotoxin gene. The Lymphotoxin sequence was retrieved from Genbank (M16441) and contained an A to G SNP transition at position 1069. Primer sequences for use in PCR amplification comprised the sense strand nucleotide sequence, 5′-CTTCTCTGTCTCTGACTCTCCATC-3′ (SEQ ID No: 22) and the reverse strand nucleotide sequence, biotin-5′-CAAGGTGAGCAGAGGGAGAC-3′ (SEQ ID No: 23)(i.e., the reverse strand was biotinylated).

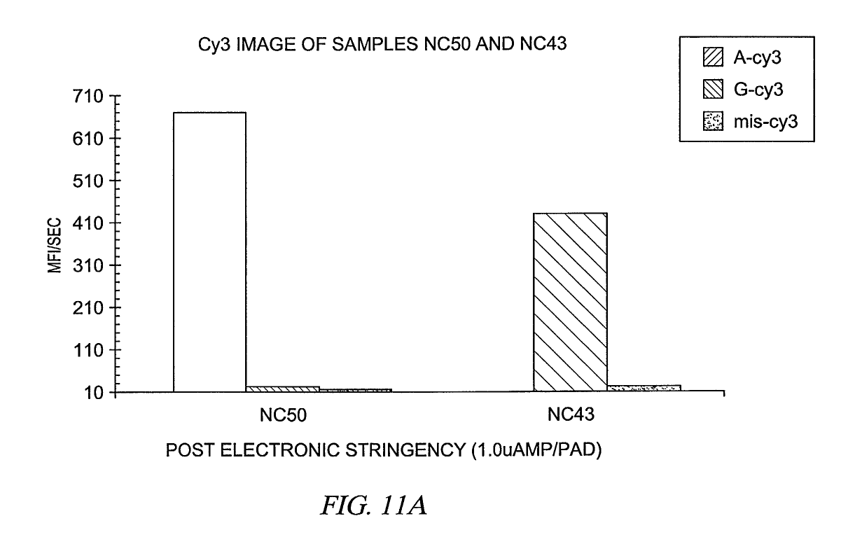

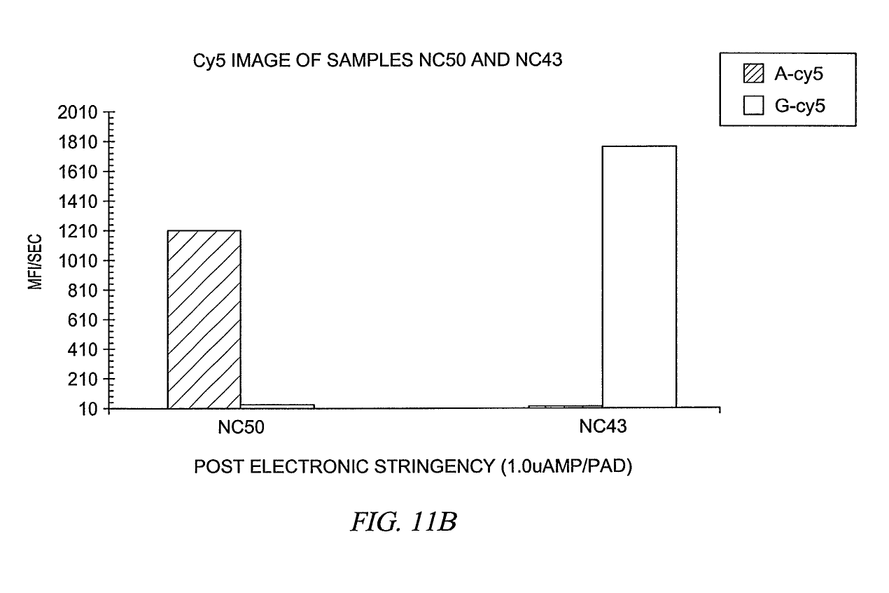

In this example, the cy3 and cy5 reporter groups were designed to identify genetic variants containing a single nucleotide polymorphism of an A or a G at position 1069 in the Lymphotoxin gene (reporter oligo synthesis by Bioserve, Laurel, Md.). (see Table 4). Loss of fluorescence following increased electronic stringency illustrated the denaturation of the non-A/non-G mismatched (mis-cy3) reporters from test sites containing the known homozygous A/A biotinylated amplicon for sample NC50 and the known homozygous G/G biotinylated amplicon for sample NC43 (FIGS. 11A and B). Consequently, the amplicons from samples NC50 and NC43 known to be A/A and G/G homozygotes, respectively, by sequencing technology were similarly scored as A/A and G/G homozygotes, respectively, by the electronic format.

TABLE 4

| | |
|---|---|
| reporter specificity | reporter sequence |

| | | |
|---|---|---|
| allele A | 5′ttctgccatgAttcctctctg3′ | (SEQ ID No: 24) |
| allele G | 5′ttctgccatgGttcctctctg3′ | (SEQ ID No: 25) |
| mismatch - T | 5′ttctgccatgTttcctctctg3′ | (SEQ ID No: 26) |
| mismatch - C | 5′ttctgccatgCttcctctctg3′ | (SEQ ID No: 27) |

### EXAMPLE 4

### Tumor Necrosis Factor α Detection

Yet, another example demonstrating the application of the invention to detect SNPs was observed in the promoter for the Tumor Necrosis Factor gene. The Tumor Factor Necrosis genomic DNA sequence was retrieved from Genbank (X02910) and contained a G to A SNP transition at position 308. Primer sequences for use in PCR amplification comprised the sense strand nucleotide sequence, 5′-GTTAGAAGGAAACAGACCACAGACC-3′ (SEQ ID No: 28) and the reverse strand nucleotide sequence, biotin-5′-TCCTCCCTGCTCCGATTCC-3′ (SEQ ID No: 29)(i.e., the reverse strand was biotinylated).

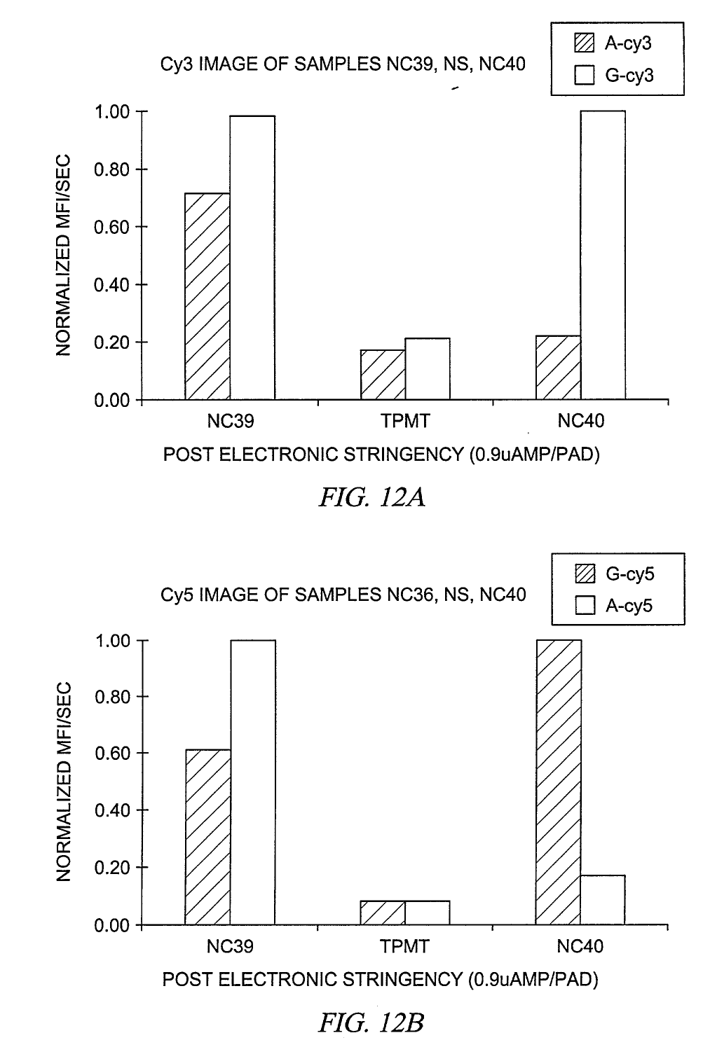

In this example, the cy3 and cy5 reporter groups were designed to identify genetic variants containing a single nucleotide polymorphism of a G or an A in the promoter for the Tumor Factor Necrosis gene (reporter oligo synthesis by Bioserve, Laurel, Md.). (see Table 5). In a modified version of the invention, only two reporter groups were used to detect either the G or A allele. Electronic stringency was applied to test sites containing amplicons from samples NC39 and NC40 as well as the non-specific amplicon, TPMT. The electronic stringency revealed that the known A/G heterozygote as determined by DNA sequencing was an A/G heterozygote for sample NC39. Moreover, the known G/G homozygote as determined by DNA sequencing was identified as a G/G homozygote for sample NC40 (FIG. 12A). For scoring purposes, the remaining electronically hybridized reporter signals were normalized to the greatest MFI/sec and the results were compared to that for the non-specific control TPMT. This example demonstrates not only the application of the invention in identifying SNPs, but also screening methodology which reduces the number of reporter signals necessary to identify specific zygosity.

TABLE 4

| | |
|---|---|
| reporter specificity | reporter sequence |

| | |
|---|---|
| Allele G | 5′gcatgGggacggggttc3′ (SEQ ID No:30) |
| allele A | 5′gcatgAggacggggttc3′ (SEQ ID No:31) |

### EXAMPLE 5

### Multiplex Analysis

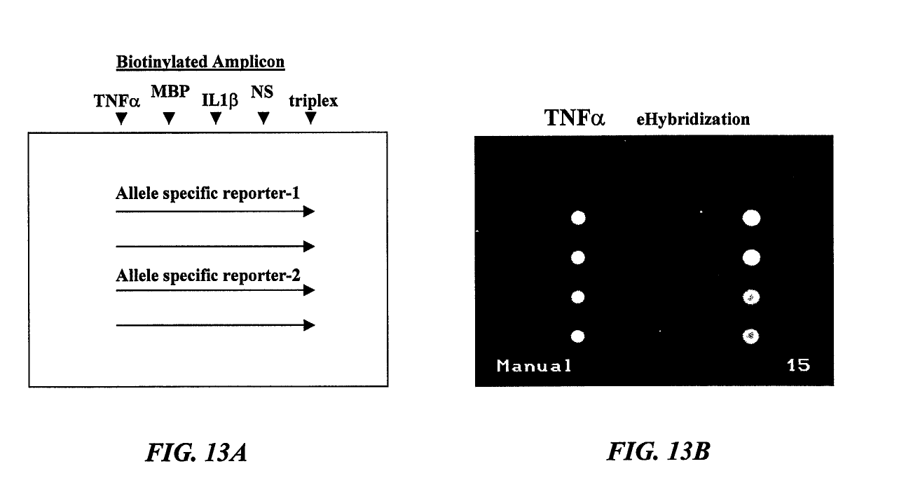

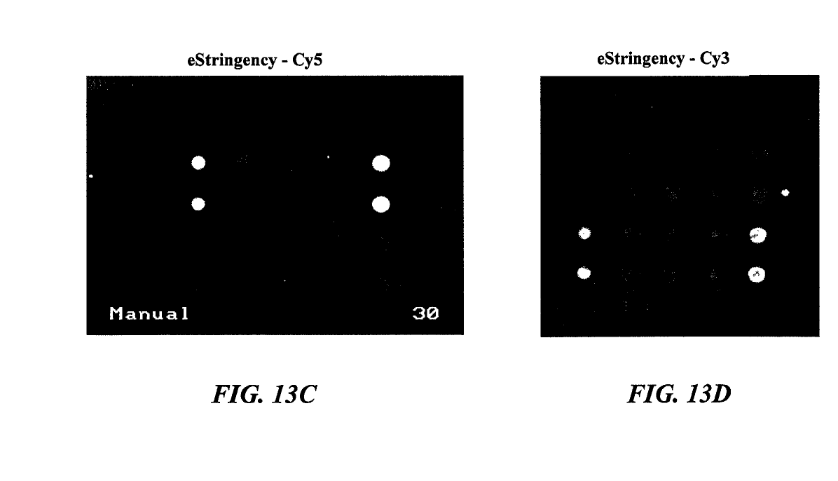

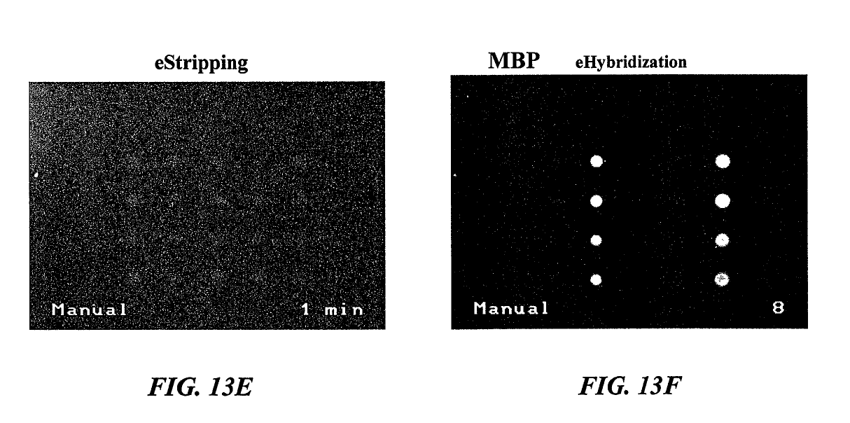

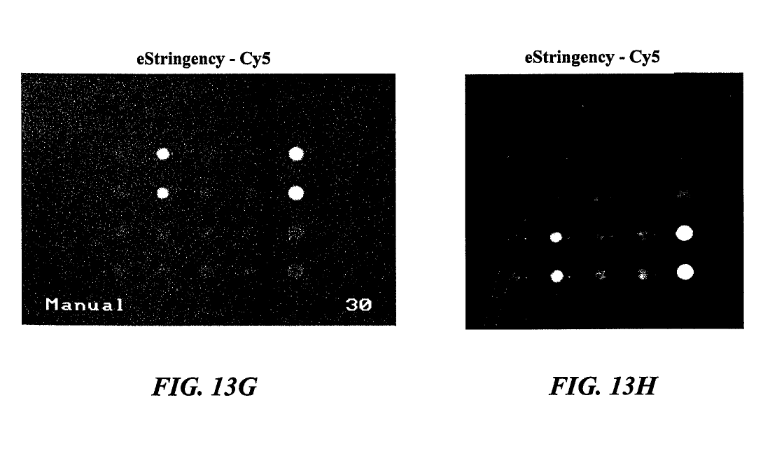

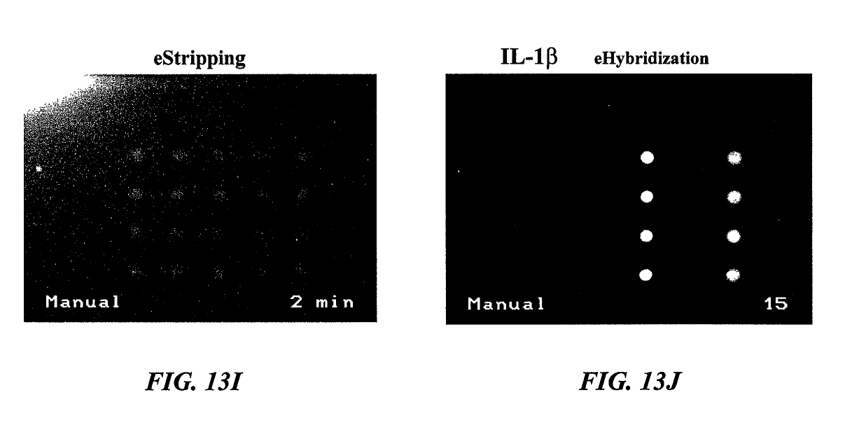

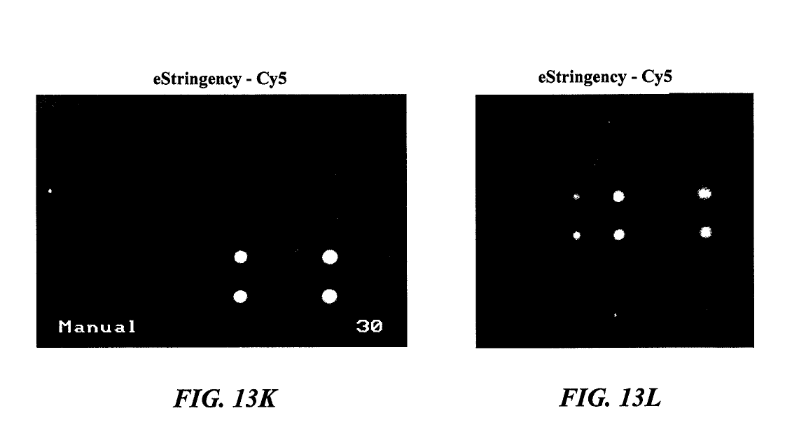

Experiments have also shown SNP discrimination from a TNFa, MBP, and IL-1b multiplexed sample in which the microchip was electronically stripped and reprobed for the genotyping of the subsequent genes in the triplex. Three amplicons, the MBP A/A homozygote (M), the IL-1β T/T homozygote (I), and the TNFα G/G homozygote (T), were capture loaded individually and as a 1:1:1 triplex mixture (T+M+I) on a 25 pad solution process chip (FIG. 13A). The chip was hybridized in duplicate with TNFα specific Cy3/Cy5 labeled reporter groups (13B). The allele specific polymorphism was obtained by electronic stringency applied in increased increments (13C and D). After the genotype of TNFα in the multiplex was obtained, the chip was stripped of labeled reporter by the application of electronic stringency at an elevated temperature (42° C.) followed by a 2 minute wash in 0.5×SSC, pH 11.5. The stripped chip was then extensively washed in histidine and a baseline imaged obtained (13E). The chip was then re-hybridized with MBP allele specific Cy3/Cy5 reporter sets (13F) and the genotype was obtained by application of electronic stringency (13G and H). The genotyped chip was electronically stripped (again), a baseline image was obtained (13I) and IL-1β allele specific reporter groups were hybridized to the multiplex (13J). The genotype was obtained by electronic stringency (13K and L). These results demonstrate that the throughput parameters of the electronic dot blot assay may be increased by the capacity to genotype multiple genes on pads which require a single capture loading event.

As shown above multiplexing of samples can be accomplished in several ways. In one format, multiple targets obtainable from a single patient sample can be tested on a single open array microchip. Further, each target may be captured on either separate locations on the microchip or on the same capture pad. Multiplexing may also accommodate multiples of patient samples on a single microchip array. In this situation, the individual target species of each patient may be captured for analysis on either separate capture sites or on groups or single sites for each patient.

The foregoing is intended to be illustrative of the embodiments of the present invention, and are not intended to limit the invention in any way. Although the invention has been described with respect to specific modifications, the details thereof are not to be construed as limitations, for it will be apparent that various equivalents, changes and modifications may be resorted to without departing from the spirit and scope thereof and it is understood that such equivalent embodiments are to be included herein. All publications and patent applications are herein incorporated by reference to the same extent as if each individual publication or patent application was specifically and individually indicated to be incorporated by reference.

We claim:

1. A method for detecting at least one single nucleotide polymorphism in at least one patient nucleic acid containing sample using an electronically addressable microchip having a plurality of test sites, wherein each test site comprises an individually controllable electrode covered by a permeation layer, the method comprising:
i. providing at least one sample nucleic acid containing at least one target nucleic acid sequence of interest that contains at least one single nucleotide polymorphism locus;
ii. electronically biasing one or more specified test sites on the microchip in order to concentrate the sample nucleic acid at the specified test sites;
iii. immobilizing the sample nucleic acid onto the test sites;
iv. electronically hybridizing a mixture comprising first and second differently labeled nucleic acid probes to the immobilized sample nucleic acid to form hybridized complexes, wherein the first probe contains a sequence specific for one allele of the single nucleotide polymorphism of the locus, and the second probe contains a sequence differing from the first sequence by a single base;
v. performing electronic stringency on said hybridized complexes to destabilize hybridization complexes comprising at least one mismatch between the immobilized sample nucleic acid and the hybridized probe; and
vi. detecting hybridization complexes that remain following the electronic stringency step (v) by detecting the hybridized labeled probes.

2. The method of claim 1, further comprising the step of subjecting the sample nucleic acid containing the target nucleic acid sequence in the patient sample to an amplification reaction whereby the amount of sample nucleic acid containing the target nucleic acid sequence is increased.

3. The method of claim 1 wherein the single nucleotide polymorphism locus is bi-allelic.

4. The method of claim 1 wherein the single nucleotide polymorphism locus is multi-allelic.

5. The method of claim 1 wherein at least one target nucleic acid sequence of interest is selected from the group consisting of whole or partial naturally occurring nucleic acid sequences encoding (1) mannose binding protein, (2) Fc-gamma receptors, (3) major histocompatibility complex proteins, (4) Interleukin 1β, (5) lymphotoxin, and (6) tumor necrosis factor α.

6. The method of claim 1 further comprising providing at least one control target nucleic acid, wherein the control target nucleic acid contains a sequence specific for an allele of the single nucleotide polymorphism locus, and wherein the control target nucleic acid is immobilized at a selected test site.

7. The method of claim 2 wherein the sample nucleic acid further comprises a biotin moiety, and the sample nucleic acid is immobilized on the selected test sites by a biotin-streptavidin interaction.

8. The method of claim 1 wherein the electronic stringency step (v) is carried out until the detectable signal from hybridization complexes comprising at least one mismatch between the immobilized target nucleic sequence and the hybridized probe is significantly reduced as compared to the detectable signal from hybridization complexes without any mismatch between the immobilized target nucleic sequence and the hybridized probe.

9. The method of claim 8 wherein the electronic stringency step (v) is carried out until the detectable signal from hybridization complexes comprising at least one mismatch between the immobilized target nucleic sequence and the hybridized probe is approximately the same as a background signal.

10. The method of claim 8 further comprising monitoring the detectable signal from the from hybridization complexes between the immobilized target nucleic sequence and the hybridized probes with a monitoring device in real time various stages of electronic hybridization and electronic stringency in order to determine when the detectable signal from hybridization complexes comprising at least one mismatch between the immobilized target nucleic sequence and the hybridized probe is significantly reduced.

11. The method of claim 10 wherein a controller for the power level or length of time of the electronic stringency applied in step (v) is functionally connected to a microprocessor which is functionally connected to the monitoring device, whereby the extent of the electronic stringency applied in step (v) is reduced or ceased in response to the detection of a significantly reduced detectable signal.

12. The method of claim 1 wherein the first and second nucleic acid probes both contain a sequence specific for an allele of the single nucleotide polymorphism locus.

13. The method of claim 1 wherein the first and second nucleic acid probes are differently labeled with different fluorophores.

14. The method of claim 13 wherein the different fluorophores are Cy3 and Cy5.

15. The method of claim 1 wherein a plurality of sample nucleic acids from each patient sample are immobilized on one test site, and wherein each sample nucleic acid contains a target nucleic acid sequence containing a different single nucleotide polymorphism locus.

16. The method of claim 1 wherein a plurality of sample nucleic acids from each patient sample are immobilized on a selected group of test sites, wherein each sample nucleic acid is immobilized on at least one separate test site, and wherein each sample nucleic acid contains a target nucleic acid sequence containing a different single nucleotide polymorphism locus.

17. The method of claim 1 wherein a plurality of patient samples are provided and immobilized on the microchip, and wherein sample nucleic acids from each patient sample are immobilized on a different set of selected test sites on the microchip during steps (iv) through (vi).

18. The method claim 17 wherein said patient sample nucleic acids are sequentially immobilized onto the test sites by repeating steps (ii) and (iii) for each patient sample.

19. The method of claim 1 wherein step (iv) is repeated with at least one additional mixture of first and second probes.

20. The method of claim 1 further comprising the steps of:
(a) stripping all hybridized probes from the immobilized sample nucleic acids using a method selected from the group consisting of electronic denaturing, chemical denaturing, thermal denaturing, and combinations thereof; and
(b) repeating steps (iv) through (vi) with at least one additional mixture of first and second probes.

21. The method of claim 20 wherein the patient's genotype for more than one single nucleotide polymorphism locus is determined from the data gathered in the multiple steps (vi).
